(19) United States
(12) Patent Application Publication
(10) Pub. No.: US 2020/0113725 A1
(43) Pub. Date: Apr. 16, 2020

(54) A Postural Feedback and Support System

(71) Applicant: Strong Arm Technologies, Inc., Brooklyn, NY (US)

(72) Inventors: Sean Petterson, New York, NY (US); Michael Kim, New York, NY (US); Michael Spinelli, New York, NY (US); Kyung In Noh, Brooklyn, NY (US); Timothy Bromm, Brooklyn, NY (US)

(21) Appl. No.: 16/060,817

(22) PCT Filed: Dec. 8, 2016
(86) PCT No.: PCT/US16/65638

§ 371 Date: Sep. 20, 2018

Related U.S. Application Data
(60) Provisional application No. 62/265,338, filed on Dec. 9, 2015.

(51) Int. Cl. A61F 5/02

(52) U.S. Cl. CPC A61F 5/024; A61B 5/486; A61F 5/026

(57) ABSTRACT

Embodiments of the present disclosure relate to an exoskeleton support system for posture feedback and support during body trunk motions. The system comprises a biomimetic semi-rigid support system connected to body attachment straps for coupling the system onto a user. A feedback pad is positioned on the user's back at a location corresponding to a middle thoracic region of the user. This arrangement causes the user to experience pressure feedback on the back when torso flexion and twisting occur. In addition, progressive resistance on the shoulders is provided via shoulder straps containing stitching in a curved manner to allow for longitudinal flexion and unrestricted arm motion during system use. Further, the system provides indications of safe movements by way of little to no feedback during neutral torso positions. Accordingly, the system only delivers feedback pressure in response to an amount of the user's own body motion.

### RELATED APPLICATION

This application claims the benefit of U.S. Provisional Application No. 62/265,338, filed on Dec. 9, 2015. The entire teaching of the above application is incorporated, in its entirety, herein by reference.

### BACKGROUND

According to the Centers of Disease Control and Prevention (CDC), back injuries account for 20% of all workplace injuries, costing the United States, alone, roughly $20-50 billion per year. In fact, back injury is the single largest category of injury according to the CDC.

Current preventative approaches to prevent back injury in the workplace involve the use of back-belts. Unfortunately, studies that have analyzed workplace use of back-belts were limited in nature and, as such, the results of these studies are not able to support the effectiveness of back-belts in injury reduction. Moreover, back-belts don't reduce problems with workers themselves (i.e., due to individual risk factors). For example, a worker who uses improper body mechanics is not trained to use proper body mechanics by simply wearing a back-belt.

### SUMMARY

The present disclosure relates an exoskeletal support system which can be worn on the back of a subject (e.g., a worker) to deliver instruction when potentially high risk torso movements are occurring; while remaining passive during static periods of the subject's trunk. These and other aspects of the present disclosure are accomplished by a device configured to be coupled to the subject's back. The device comprises a semi-rigid feedback member that is configured to deliver instructional feedback to the subject. The semi-rigid feedback member can be supported on the subject via dynamic attachment straps.

The intensity of instructional feedback delivered can be adjusted using several means, including the pre-tensioning adjustment of dynamic, fabric shoulder straps. Additional, yet independent, tunability of the instructional feedback can be accomplished through a leaf spring element attached in series with the semi-rigid feedback member that can be adjusted using a sliding mechanism and locking dial.

An object of the present disclosure is to provide pressure instruction in the lumbar and thoracic regions of a person's spine and shoulders when a desired amount of torso flexion or torso twisting occurs.

A further object of this disclosure is to maintain separation between the feedback member and the person's torso during periods of less-risky torso trunk movements. This is accomplished using a biomimetic curvature of the semi-rigid feedback member. Further, a position and height of the semi-rigid feedback member is adjusted using a spring adjustability mechanism such that the semi-rigid feedback member is continually form fitted/aligned with the subject's spinal curvature.

A further object of the present disclosure is to allow full range of arm and shoulder motions while maintaining feedback capabilities. This is accomplished with a multi-layer fabric strap system with stitch lines placed longitudinally along the straps and in an undulating pattern around the shoulders to allow for flexibility in the lateral direction during shoulder and arm movements while still providing the feedback capabilities during potentially high-risk motions.

The combined structural configuration of each element of the device is what enables delivery of instructional feedback to the subject in response torso trunk movements. The structural configuration only provides instructional feedback for body movement that can cause stress injury. In fact, the device remains passive (i.e., provides little to no feedback pressure) during periods of inactivity or less-risky motions.

Aspects of the present disclosure provide a postural feedback support system for a person. The system further comprises a semi-rigid feedback member including an upper end and a lower end. The semi-rigid feedback member has a length corresponding to a spine of the person. In addition, the system includes at least one body attachment strap mechanically coupled to the upper end of the semi-rigid feedback member. The at least one body attachment strap is configured to secure the semi-rigid feedback member to the person. The system also includes a postural feedback pad coupled to a person-facing side of the semi-rigid feedback member. The postural feedback pad is at a location on the semi-rigid feedback member that corresponds to a middle thoracic region of the person. In that arrangement, the system is configured to provide instructional feedback to the person in response to high risk torso movements of the person by way of the semi-rigid feedback member and the postural feedback member. In this case, the high risk torso movements are movements that are associated with a likelihood of back injury.

In certain embodiments, the semi-rigid feedback member has a biomimetic curvature corresponding to a spine of the person having a standing balanced posture. The semi-rigid feedback member can be configured to flex in response to torso movements of the person. Additionally, the semi-rigid feedback member can be further configured to provide the instructional feedback in the form of physical pressure feedback, via the postural feedback pad, onto the middle thoracic region of the person.

In other aspects, the system can further comprise a height adjustment housing configured to receive the lower end of the semi-rigid feedback member. The height adjustment housing can be configured to enable adjustment of a height of the semi-rigid feedback member with respect to a torso of the person. The height adjustment housing can be further configured to receive a waist belt comprising two belt halves, each having terminal ends, the terminal ends thereof configured to fasten with one another.

In further embodiments, the lower end of the semi-rigid feedback member defines a toothed track. The toothed track is configured to cooperate with a spring-loaded lock-and-pin mechanism. The lock-and-pin mechanism engages with the toothed track to adjust a height of the semi-rigid feedback member with respect to a torso of the person.

In certain embodiments, the system can further comprising a flexibility adjustment housing mechanically coupled the upper end of the semi-rigid feedback member. An upper housing can be in pivotal engagement with the flexibility adjustment housing. Additionally, the upper housing can be configured to receive the at least one body attachment strap. Further, a flexible element can be in mechanical cooperation with the flexibility adjustment housing and the upper housing to provide restrictive feedback to the person via the at least one body attachment strap.

In some aspects, the flexible element can include a first end, a second end, and a longitudinal axis extending between the first end and the second end. The first end of the flexible element can be coupled to the upper housing and the second end of the flexible element can be coupled to the flexibility adjustment housing such that the upper housing is in pivotal engagement with the flexibility adjustment housing.

In additional embodiments, the system can further comprise a stiffening element in slidable engagement with the flexible member. A stiffening adjustment mechanism can be configured to interface with the stiffening element to actuate the stiffening element along the longitudinal axis of the flexible element. The stiffening element and the stiffening adjustment mechanism can be configured to form a rack and pinion mechanism. The stiffening adjustment mechanism can be housed by the flexibility adjustment housing.

A position of the stiffening element with respect to the longitudinal axis of the flexible element can correspond to a level of flex of the flexible element. For example, a position of the stiffening element that is at or near the first end of the flexible element can correspond to a most rigid state of the flexible element. Similarly, a position of the stiffening element that is at or near the second end of the flexible element can correspond to a most flexible state of the flexible element. Accordingly, an amount of restrictive feedback provided to the person via the at least one body attachment strap can be a function of the level of flex of the flexibility element.

In additional aspects, the at least one body attachment strap comprise curved stitching to enable longitudinal flexion of the at least one body attachment strap and unrestricted motion of the person's arms.

### BRIEF DESCRIPTION OF THE DRAWINGS

The foregoing will be apparent from the following more particular description of example embodiments of the disclosure, as illustrated in the accompanying drawings in which like reference characters refer to the same parts throughout the different views. The drawings are not necessarily to scale, emphasis instead being placed upon illustrating embodiments of the present invention.

FIG. 1 is a perspective view of an exoskeleton posture support system that is coupled to a person's back via shoulder straps and a waist belt, in accordance to an example embodiment of the present disclosure.

FIG. 2 is an exploded perspective view of an upper housing and a flexibility housing of an exoskeleton posture support system according to an example embodiment of the present disclosure.

FIG. 3 is an exploded perspective view of a height adjustability housing of an exoskeleton posture support system according to an example embodiment of the present disclosure.

FIG. 4 is a user-facing perspective view a lower portion of an exoskeleton posture support system in accordance with an example embodiment of the present disclosure.

FIG. 5 illustrates an example body attachment shoulder strap for use with an exoskeleton support system.

FIG. 6 illustrates an example waist belt for use with an exoskeleton support system.

### DETAILED DESCRIPTION

A description of example embodiments of the disclosure follows.

Injuries relating to the human back and upper extremities have been known to be related to torso movements and excessive flexion of the human back. In many cases an injury occurs during unsafe body movement without any prior bodily indication or warning. Existing research has shown certain trunk movements including torso flexion and twisting are more likely to cause back injury. In contrast, movements that involve more neutral spinal positions have been shown to be less likely to result in back injury. However, research has indicated that even professional training on proper movement techniques designed to reduce injury are not lasting in their effect due to human tendencies resorting to unsafe movements over time.

Prior attempts at mechanical postural feedback typically rely on continuous pressure around the torso region. While accomplishing torso pressure at all time, this method fails to convey a differentiation between safe and unsafe torso positions, thus removing any indication to the user when specifically unsafe movements are occurring. As a result any potential behavior alteration effects from the user are essentially non-existent.

Solutions utilizing electronics for pressure or notification have also been presented for this problem. However the need for power and electronics creates design complications that are often prohibitive in industrial or work settings, and creates limitations to the device based on battery life and environment. Furthermore, there exists user reluctance to adopt technology that includes electronics coupled to the human body, which also limits the effectiveness of these concepts.

Therefore, there is a need for an on-body feedback system only when excessive torso movements occur. Such a system is valuable to prevent workplace injuries for those performing manual tasks who are either not aware of safer practices (e.g., body mechanics), or who are not consistent in performing moderate movements.

Particular advantage is gained through embodiments of the present disclosure which utilize a user's own body movements to apply a physical pressure reminder only when dangerous positions occur, and which does not apply pressure during periods of inactivity or moderate movement. Additionally, embodiments of the present disclosure provide users with complete control over feedback adjustability. Further, users are delivered instant notification with respect to a level of safety corresponding to their movements. Embodiments of the present disclosure provide this notification via a mechanical response to natural physical mechanical movement of the user's body. The mechanical response occurs without the use of a power supply.

In an exemplary aspect, disclosed is an exoskeletal support system that is a wearable unpowered exoskeletal support system which can be worn on the back of a subject to deliver instructional feedback when potentially high risk torso movements are occurring while remaining passive during static periods of the subject's trunk.

The system features a rigid biomimetic external spine that fits on the subjects back side. The external spine includes an adjustment feature that allows for variable rigidity. This external spine can be fastened onto the body of the subject with shoulder straps similar to a backpack and a waist belt. The external spine can have an adjustable height column to fit various different heights of the subject (e.g., their torso). Similarly, the shoulder straps and waist belts can be adjusted to the users comfort. Between the external spine and the subjects back, there can be two pads that touch the lower lumbar region (located on the deepest curve of your lower back) and the middle thoracic region.

With the system properly worn on the body, subjects are provided physical feedback based on their own movements. For example, when the subject bends over in flexion to pick something up with a curved back without bending their knees (a posture or form of lifting commonly described to explain improper lifting technique that may result in lower back injury), the external rigid spine bends or flexes, resisting the curved back, and results in a physical pressure feedback through the middle pad and onto the subjects middle thoracic region. Through this feedback subjects are conditioned and encouraged to lift in such a way that avoids this resistance or feedback by keeping their back straight and compensating by bending at their knees and lifting with their legs. Similar to resistance in forward bending, the exoskeletal system is designed to help reduce over twisting of the torso (i.e., another posture or form of lifting commonly described to explain improper lifting technique that may result in lower back injury). This is accomplished with the shoulder straps which applies uniform pressure across the shoulders when a subject over rotates. The straps are designed in a way to provide this pressure and feedback while minimizing the effects of chafing and an uncomfortable sensation.

The exoskeletal system of the present disclosure differs from various existing solutions in that it accomplishes postural feedback through an unpowered wearable device which harnesses the subject's own body motions to apply pressure in the back region in a self-correcting feedback system. Furthermore, muscle atrophy can occur with other systems due to constant or excessive pressure to the back region of a user. With the curved, biomimetic nature of the present support system, postural feedback pressure can be eliminated during periods of safe body activity. This functions to prevent the muscle atrophy that often occurs using other feedback devices. This interconnected nature of the device feedback in relation to the user's own body motion creates a safer, more connected experience not achieved with other existing solutions.

The biomimetic support structure of the disclosed exoskeletal system also allows for a slim on-body profile applicable in tight-space situations and functions that involve partial sitting. Additional adjustment features in the waist straps and height adjustment allow the device to be utilized across a wide range of body shapes encountered in industrial and work settings. Intuitive design features including the shoulder attachment straps and adjustment mechanisms allow the device to be quickly taken on and off for more effective use during in field applications and allowing for easier adoption by users.

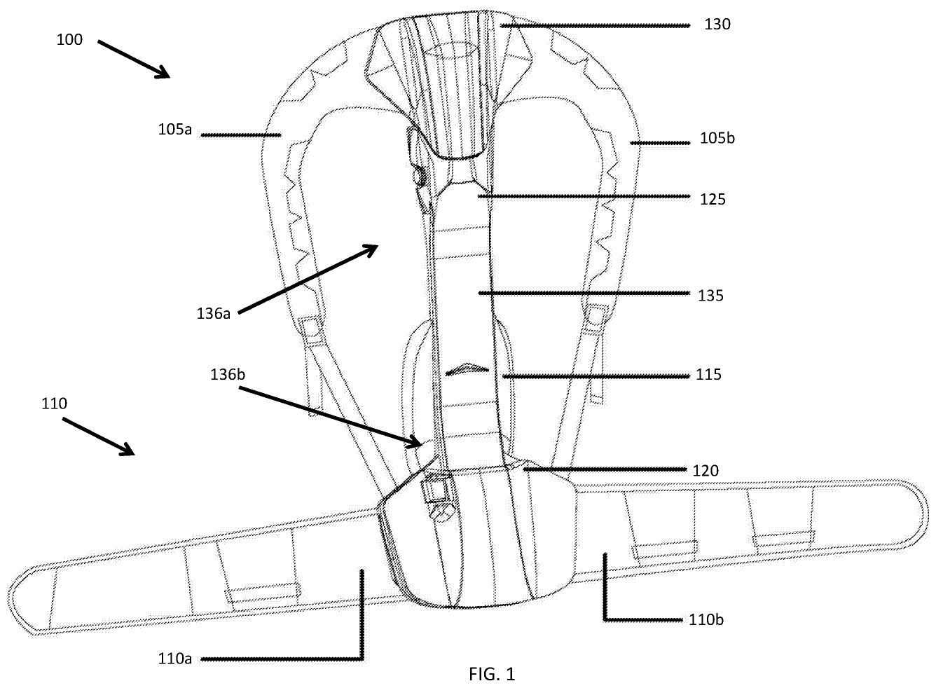

FIG. 1 is a perspective view of an exoskeletal posture support system 100 (e.g., a postural feedback support system) that can be coupled to a person via shoulder straps 105a-b and a waist belt 110a-b, in accordance to an example embodiment of the present disclosure. The shoulder straps 105a-b can be adjustable, fabric shoulder straps, much like shoulder straps on a backpack. Additionally, the waist belt 110a-b can be fastened around the person's waist using two, hook-and-loop, overlapping waist belt attachment flaps 110. The exoskeleton posture support system 100 is designed to use pressure from a postural feedback pad 115 and restriction from the shoulder straps 105a-b as instructional feedback for manual laborers to indicate improper body mechanics (e.g., improper lifting techniques).

The exoskeletal posture support system 100 includes a semi-rigid feedback member 135. The semi-rigid feedback member 135 includes an upper end 136a and a lower end 136b. The semi-rigid feedback member of 135 has a length that corresponds to the person's spine. Additionally, the semi-rigid feedback member 135 has a biomimetic curvature. The biomimetic curvature corresponds to a curvature of the person's spine. For instance, the biomimetic curvature corresponds to the person's spine when the person has a standing balanced posture. Accordingly, the semi-rigid feedback member 135 is configured to flex in response to torso movements of the person. In particular, the semi-rigid feedback member 135 is configured to flex in response to high risk torso movements. The high risk torso movements are defined as movements that are known to be associated with a likelihood of back injury.

In response to the high risk torso movements, the semi-rigid feedback member 135 provides instructional feedback to the person. For example, the instructional feedback can be in the form of physical pressure feedback that is provided to the person via the postural feedback pad 115. The postural feedback pad 115 directs the physical pressure feedback onto the middle thoracic region of the person.

The postural feedback pad 115 is coupled to a person facing side of the semi-rigid feedback member 135. In particular, the postural feedback pad 115 is positioned on the semi-rigid feedback member 135 at a location that corresponds to the middle thoracic region of the person. In particular, the postural feedback pad 115 is designed to help prevent lower back injuries by acting as a physical reminder to the person when the person is in the wrong posture while performing manual labor tasks.

A flexibility adjustment housing 125 is mechanically coupled to the upper end 136a of the semi-rigid feedback member 135. The flexibility adjustment housing is configured to enable the person to adjust an amount of instructional feedback provided to the person. In particular the flexibility adjustment housing 125 is configured to enable the user to adjust an amount of instructional feedback provided to the person shoulders by way of body attachment straps (e.g., shoulder straps) 105a-b. The body attachment straps 105a-b are mechanically coupled to an upper housing 130, which is pivotally engaged with the flexibility adjustment housing 125. This upper housing 130 is designed to sit closely to the upper back of the person.

Further, the postural feedback support system 100 includes a height adjustment housing 120 that is configured to receive a lower end 136b of the postural feedback support system 100. The height adjustment housing 120 is configured to enable the person to adjust a height of the semi-rigid feedback member 135. In particular, the height adjustment housing 120 enables a user to adjust the height of the semi-rigid feedback member 135 in order to ensure that the biomimetic curvature and length of the semi-rigid feedback member 135 correspond to the person spine. Further, the height adjustment housing 120 is coupled to a waist belt 110 comprising two waist belt halves 110a-b. The waist belt 110 is configured to secure the postural feedback support system 100 around the person's waist.

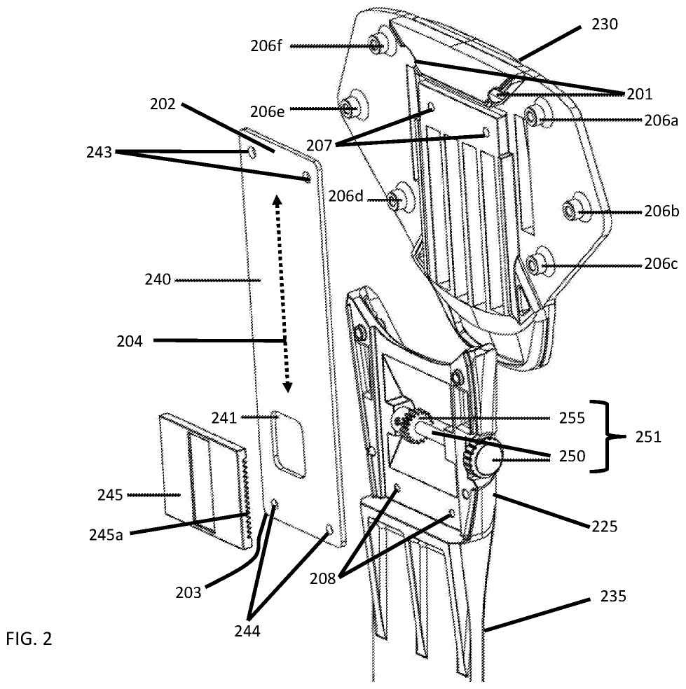

FIG. 2 is an exploded perspective view of an upper housing 230 and a flexibility housing 225 of an exoskeletal posture support system (e.g., the postural feedback support system 100 of FIG. 1). As illustrated, a flexibility adjustment housing 225 is coupled to an upper end (e.g., the upper end 136a of FIG. 1) of a semi-rigid feedback member 235. The flexibility adjustment housing 225 is in pivotal engagement with the upper housing 230. In particular, a flexible element 240 is in mechanical cooperation with the flexibility adjustment housing 225 and the upper housing 232 to enable pivotal engagement there-between. In some examples, the flexibility element 240 is made of a composite material.

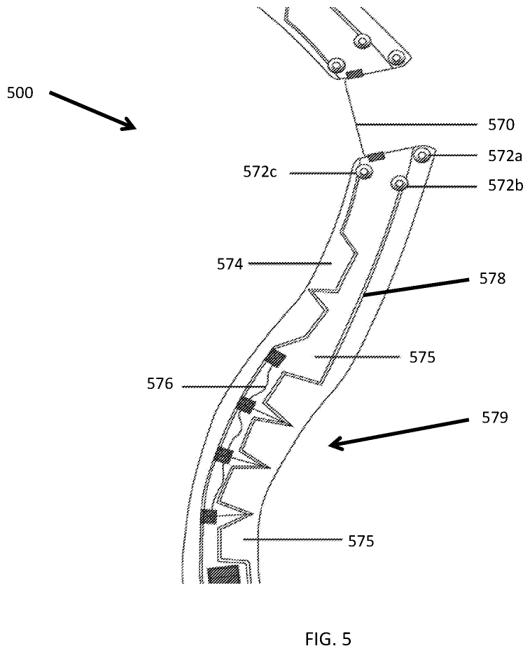

The upper housing 230 includes housing posts 206a-f. The housing posts 206a-f can be molded into the upper housing 230 itself. The housing post 206a-f are configured to interface with attachment straps grommets (e.g., grommets 572a-c of FIG. 5) to enable coupling of body attachment straps (e.g., the body attachment straps 105a-b of FIG. 1) to the upper housing 230. Additionally, the housing posts 206a-f are configured to act as bolt holes to bolt on an upper housing encasing (not shown) to the upper housing 230.

The flexibility element 240 is secured to the upper housing, at a first end 202, via a fastener (e.g., a threaded screw or a bolt) that is inserted into through holes 243 and received by threaded inserts 207. Also, the flexibility element 246, at a second end 203, is secured to the flexibility adjustment housing 225 via another fastener that is inserted into through holes 244 and received by threaded inserts 208.

A stiffening element 245 is in slidable engagement with the flexibility element 240. In particular, stiffening element 245 is configured to interface with stiffening adjustment mechanism 251 via housing portal 241 defined at the second end 203 of the flexibility element 240. The stiffening element 245 is actuated along a longitudinal axis 204 of the flexibility element 240 in response to a user's interaction with the stiffening adjustment mechanism 251. The longitudinal axis 204, as illustrated, extends between the first end 202 and the second end 203 of the flexibility element 240. In this example, the stiffening adjustment mechanism 251 forms a rack and pinion mechanism. In particular, a dial-and-post 250 is mechanically coupled to a gear 255. Accordingly, in response to a user turning the dial-and-post 250, the gear 255 actuates the stiffening element 245 along the longitudinal axis 204 of the flexibility element 240 in a direction corresponding to a direction of circumferential turn of the dial-and-post 250. In particular, the gear 255 interacts with teeth 245a in order to actuate the stiffening element 245 along the longitudinal axis 204.

Accordingly, the stiffening element 245 is configured to adjust a point along the longitudinal axis 204 of the flexibility element 240 in which the flexibility element 240 can flex. Thus, as the stiffening element 245 is actuated towards the first end 202 of the flexibility element 240, the flexibility element 240 becomes increasingly rigid. Conversely, as the stiffening element 245 is actuated towards the second end 203 of the flexibility element 240, the flexibility element 240 becomes increasingly flexible. As such, the position of the stiffening element 205 with respect to the longitudinal axis 204 of the flexibility element 240 corresponds to a level of flex of the flexibility element 240. In turn, the level of flex of the flexibility element 240 corresponds to a level of resistance feedback delivered through shoulder straps (e.g., the shoulder straps 105a-b of FIG. 1) to the person.

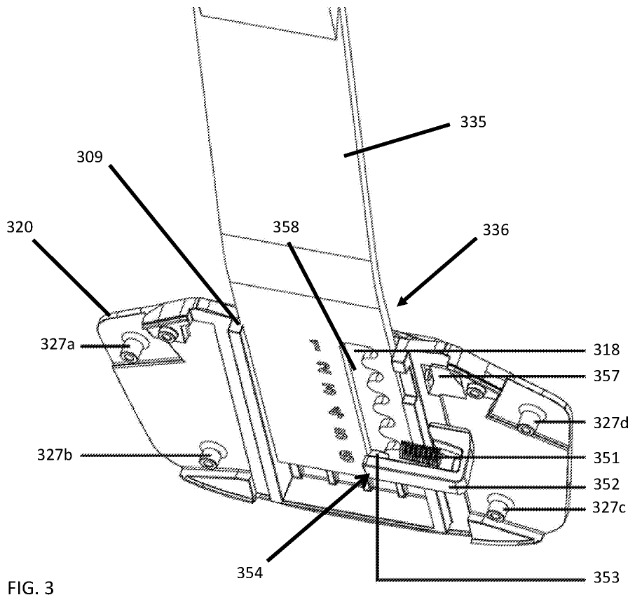

FIG. 3 is an exploded perspective view of a height adjustability housing 320 of an exoskeletal posture support system (e.g. the postural feedback support system 100 of FIG. 1). The height adjustability housing 320 is configured to receive a lower end 336 of the semi-rigid feedback member 335 of the exoskeletal posture support system. For example, the height adjustability housing 320 can receive the lower end 336 via housing track 309 which includes guide slots (not shown) for slidably receiving the lower end 336 of the semi-rigid feedback member 335.

As discussed herein, the height adjustability housing 320 is configured to enable a person to adjust a height of the semi-rigid feedback member 335. To that end, the height adjustment housing 320 includes a spring-loaded locking pin mechanism 354 that is configured to cooperate with a toothed track 318 defined by the lower end 336 of the semi-rigid feedback member 335. In particular, the spring-loaded locking pin mechanism 354 includes a height adjustment slider button that a user can compress via user interface portal 357. In response to the user compressing the slider button 352, a pin 353 coupled to an end of the slider button 352 is positioned into a longitudinal passage 358 of the track 315 such that a user may adjust the height of the semi-rigid feedback member 335. Once the desired height level is reached, the user disengages interaction with the slider button 352, causing a loaded spring 351 to position the pin 352 into a tooth of the track 318, thereby locking the semi-rigid feedback member 335 at a particular height level.

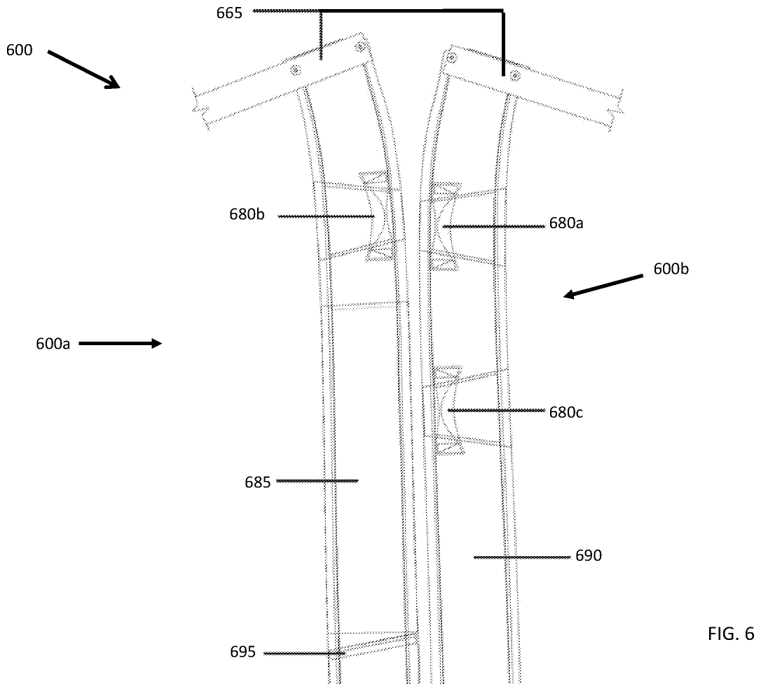

The height adjustability housing 320 includes housing posts 327a-d. The housing post 327a-d can be molded into the height adjustability housing 320 itself. The housing post 327a-d are configured to interface with grommets (e.g., grommets 665 of FIG. 6) of waist belt (e.g. the waist belt 600 of FIG. 6) and couple the waist belt to the height adjustability housing 300. Additionally, the housing posts 327a-d are configured to act as bolt holes to bolt on a housing encasing (not shown) to the height adjustability housing 320.

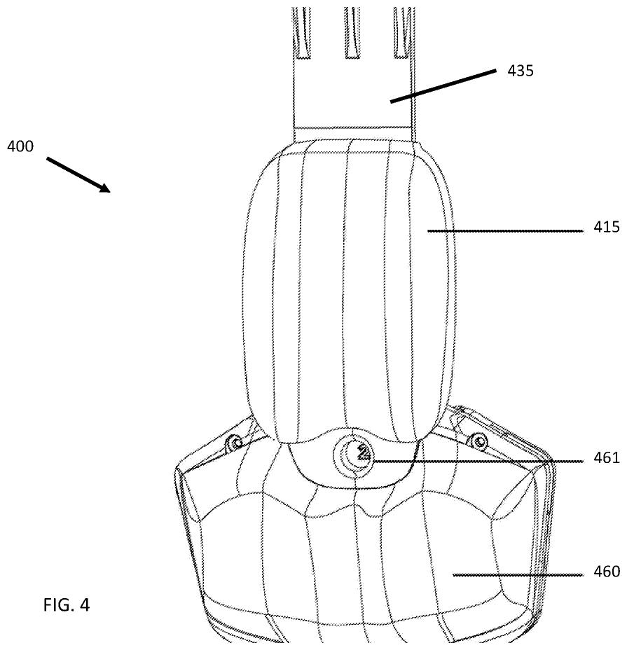

FIG. 4 is a user-facing perspective view a lower portion 400 of an exoskeletal posture support system (e.g., the posture support system 100 of FIG. 1). A postural feedback pad 415 is coupled to a person facing side of the semi-rigid feedback member 435. In particular, the postural feedback pad 415 is positioned on the semi-rigid feedback member 435 at a location that corresponds to the middle thoracic region of the person. In particular, the postural feedback pad 415 is designed to help prevent lower back injuries by acting as a physical reminder to the person when the person is in the wrong posture while performing manual labor tasks.

An eye hole 461 is defined by the housing encasing (not shown) of a height adjustability housing (e.g., the height adjustability housing 300 of FIG. 3). The eye hole 461 acts as a visual interface that allows the user to reference a number on the semi-rigid feedback member 435 that correlates directly a height of the semi-rigid feedback member 435 with respect to the user's spine.

A lumbar pad 460 is coupled to the housing encasing and is designed to add comfort and support to a lowest portion of the user's back. It rests just above a line of the user's hips.

FIG. 5 illustrates an example body attachment shoulder strap 500 for use with an exoskeleton support system. The straps are connected by a cord 570, which also acts as a hanging tool for the entire device. The cord 570 is fed through strap slot 201 of FIG. 2. The strap 500 includes grommets 572a-c, which are used to connect the strap 500 to the an upper housing (e.g., the upper housing 230 of FIG. 1) via housing posts (e.g., posts 206a-f of FIG. 2). There is a padded section 574 on the strap at a position corresponding to an area near the neck of the user. The padded section 571 is designed to provide comfort to the neck region of the user. A structural fabric 575 is designed to add structure to the strap, as well as a surrounding stitch pattern 578 that is configured to allow the strap to fold into a “U” shape. This shape allows the straps to move with and form to the parts of the human body that it touches. The strap 500 also includes utility loops 576 that can be used as optional attachment points for general user equipment such as tools. There is a padded, flexible region 575 of the strap 500 which also allows the strap to move with the user. Curved gusset stitches 579 are strategically designed to form a curve, while allowing that portion of the strap to stretch if a user is reaching or moving. It also removes any seams from the portions of the strap that touch the body, reducing the potential for discomfort.

FIG. 6 illustrates an example waist belt 600 that includes waist straps 600a-b for use with an exoskeletal support system (e.g., the postural support system 100 of FIG. 1). Each waist strap 600a-b has adjustment webbing attached (not shown) to an end of the waist straps 600a-b corresponding to the location of grommets 665. The grommets 665 also act as attachment points to the adjustment housing posts 327a-d of FIG. 3. Both waist straps 600a-b include utility loops 680a-c which are designed to be optional attachment points for general user equipment, such as tools. The left strap 600a includes a hook patch 685 which engages and overlaps with a loop patch 690 on a underside of the right strap 600b when coupling the waist belt 600 around a user's body. A pocket 695 is sewn into the hook area 685 of the left strap 600a. The pocket 695 is sized to receive the average human hand, and is used to position the belt 600 closely to the body during the process of overlapping and attaching the loop area 690 of the right strap 600b while coupling the belt 600 around the user's waist.

While this disclosure has been particularly shown and described with references to example embodiments thereof, it will be understood by those skilled in the art that various changes in form and details may be made therein without departing from the scope of the disclosure encompassed by the appended claims.

What is claimed is:

1. A postural feedback support system for a person, the system comprising:
a semi-rigid feedback member including an upper end and a lower end, the semi-rigid feedback member having a length corresponding to a spine of the person;
at least one body attachment strap mechanically coupled to the upper end of the semi-rigid feedback member, the at least one body attachment strap configured to secure the semi-rigid feedback member to the person;
a postural feedback pad coupled to a person-facing side of the semi-rigid feedback member at a location on the semi-rigid feedback member that corresponds to a middle thoracic region of the person; and
wherein the system is configured to provide instructional feedback to the person in response to high risk torso movements of the person by way of the semi-rigid feedback member and the postural feedback pad, wherein high risk torso movements are movements that are associated with a likelihood of back injury.

2. The system of claim 1, wherein the semi-rigid feedback member has a biomimetic curvature corresponding to a spine of the person having a standing balanced posture.

3. The system of claim 1, wherein the semi-rigid feedback member is further configured to flex in response to torso movements of the person and wherein the semi-rigid feedback member is further configured to provide the instructional feedback in the form of physical pressure feedback, via the postural feedback pad, onto the middle thoracic region of the person.

4. The system of claim 1 further comprising a height adjustment housing configured to receive the lower end of the semi-rigid feedback member, wherein the height adjustment housing is configured to enable adjustment of a height of the semi-rigid feedback member with respect to a torso of the person.

5. The system of claim 4, wherein the height adjustment housing is further configured to receive a waist belt comprising two belt halves, each having terminal ends, the terminal ends thereof configured to fasten with one another.

6. The system of claim 4 wherein the lower end of the semi-rigid feedback member defines a toothed track, the toothed track configured to cooperate with a spring-loaded lock-and-pin mechanism, wherein the lock-and-pin mechanism engages with the toothed track to adjust a height of the semi-rigid feedback member with respect to a torso of the person.

7. The system of claim 1 further comprising:
a flexibility adjustment housing mechanically coupled the upper end of the semi-rigid feedback member;
an upper housing in pivotal engagement with the flexibility adjustment housing, the upper housing configured to receive the at least one body attachment strap; and
a flexible element in mechanical cooperation with the flexibility adjustment housing and the upper housing to provide restrictive feedback to the person via the at least one body attachment strap.

8. The system of claim 7 wherein the flexible element comprises a first end and a second end, the flexible element further comprises a longitudinal axis extending between the first end and the second end.

9. The system of claim 8 wherein the first end of the flexible element is coupled to the upper housing and the second end of the flexible element is coupled to the flexibility adjustment housing such that the upper housing is in pivotal engagement with the flexibility adjustment housing.

10. The system of claim 9 further comprising:
a stiffening element in slidable engagement with the flexible member;
a stiffening adjustment mechanism configured to interface with the stiffening element to actuate the stiffening element along the longitudinal axis of the flexible element, the stiffening adjustment mechanism housed by the flexibility adjustment housing.

11. The system of claim 10 wherein a position of the stiffening element with respect to the longitudinal axis of the flexible element corresponds to a level of flex of the flexible element.

12. The system of claim 11 wherein a position of the stiffening element that is at or near the first end of the flexible element corresponds to a most rigid state of the flexible element.

13. The system of claim 11 wherein a position of the stiffening element that is at or near the second end of the flexible element corresponds to a most flexible state of the flexible element.

14. The system of claim 10 wherein the stiffening element and the stiffening adjustment mechanism form a rack and pinion mechanism.

15. The system of claim 11 wherein the level of flex of the flexibility element corresponds to an amount of restrictive feedback provided to the person via the at least one body attachment strap.

16. The system of claim 1 wherein the at least one body attachment strap comprised curved stitching to enable longitudinal flexion of the at least one body attachment strap and unrestricted motion of the person's arms.
